(19) United States
(12) Patent Application Publication
(10) Pub. No.: US 2004/0101550 A1
(43) Pub. Date: May 27, 2004

(54) Transdermal therapeutic system

(76) Inventors: Fred Windt-Hanke, Berlin (DE); Clemens Gunther, Berlin (DE); Reinhard Horowski, Berlin (DE); Johannes Tack, Berlin (DE)

Correspondence Address: Wood Phillips Katz Clark & Mortimer, Suite 3800, 500 West Madison Street, Chicago, IL, 60661-2511, US

(21) Appl. No.: 10/362,248

(22) PCT Filed: Aug. 24, 2001
(86) PCT No.: PCT/EP01/09824

(30) Foreign Application Priority Data
Aug. 24, 2000 (DE) 100 43 321.9

(51) Int. Cl. A61K 31/48; A61K 9/70

(52) U.S. Cl. 424/449; 514/288

(57) ABSTRACT

Use of a transdermal therapeutic system (TTS) comprising a pharmaceutical layer containing at least one matrix having an active ingredient and/or an active ingredient reservoir; a diffusion barrier that is permeable to said active ingredient and arranged on the skin side of the active ingredient reservoir; and an ergoline derivative or salt thereof as an active ingredient for producing an agent for obtaining and maintaining the circadian rhythm under dopamine therapy.

This invention relates to a transdermal therapeutic system (TTS) comprising a pharmaceutical layer containing at least one matrix having an active ingredient and/or an active ingredient reservoir; a diffusion barrier that is permeable to said active ingredient and arranged on the skin side of the active ingredient reservoir; and an ergoline derivative or salt thereof as an active ingredient for producing an agent for obtaining and maintaining the circadian rhythm under dopamine therapy.

The term “TTS” mostly denotes percutaneously acting but also transmucosal systems. A TTS typically has a sheet-like structure and is attached to an area of the skin. The system can optionally be attached to the skin by an additional skin-side adhesive that is permeable to the active ingredient. Alternatively, the matrix and/or diffusion barrier can itself have adhesive properties. And finally a non-adhesive TTS can be attached to the skin using other auxiliary means such as adhesive tapes or bandages. The matrix is a material in which the active ingredient is immobilized. An active agent in an active ingredient reservoir however is not necessarily immobilized, which is why the active ingredient reservoir must be enclosed. The diffusion barrier forms the skin-side portion of this shell. It goes without saying that all other parts of the shell should be as impermeable as possible, including diffusion paths, to the active ingredient. Immobilized means in this context that any uncontrolled active ingredient flow is prevented. However diffusion of an active agent in a matrix and/or through a diffusion barrier is not only possible but intended. The diffusion coefficients eventually determine the active ingredient flux from the TTS into a patient's skin. The dose released into a patient's skin is in first approximation a linear function of the active area of the TTS. The active area is the contact area of those TTS portions that allow active ingredient diffusion. TTSs can be used in human and veterinary medicine.

A TTS of the design mentioned above is known in principle from publication WO 92/20339. It specifically describes the effect of propylene glycol lauric acid on the flux, i.e. a considerable increase in flux. However the values specified therein relate to solutions applied to skin samples and not to the actual TTS. No specification is given regarding flux from a TTS.

A TTS containing lisuride is further known from publication WO 91/00746. The flux values for human skin samples specified therein cannot be directly transferred to any achievable in-vivo values.

TTSs of the design described above are used for various indications including Parkinson's disease. When treating Parkinson's disease, the highest possible doses are desirable. A transdermal therapeutic system also improves compliance, which is of critical importance for any combinatory treatment of this disease as patients tend to be older and have multiple diseases. Improved control and the chance to reach circadian profiles (e.g. by low stimulation as constantly as possible at night or during a break) are particularly important and have not yet been achieved (e.g. to prevent psychoses and improve the quality of sleep). The ergoline derivatives of the formula I have a partially dopamine-agonistic or partially antagonistic effect that contributes to preventing the development of psychoses and can improve existing psychoses and similar problems.

In the treatment of Parkinson's disease in which dopamine drugs and combinations thereof are taken throughout the day, concentrations in the plasma are not constant but subject to great variation, and this not only for kinetic reasons (highly variable first pass effect depending on the metabolization type) but also depending on individual administration conditions (type and times of food intake, effect of other drugs on resorption and metabolism, etc.). This is why there is a risk of temporary overdosing, which may result in REM suppression and the resulting sleep disturbances or psychoses.

In addition, currently used dopamine therapies frequently have lasting and severe side effects. This is where a transdermal therapeutic system according to the invention described below can ensure individually dosable, adjustable, and controlled action time (if required, by removing the patch) without influencing the circadian rhythm that is often disturbed as a result of treating Parkinson's disease and other dopaminergic diseases.

It is the technological problem of the invention to provide an agent for obtaining and maintaining the circadian rhythm that can be individually dosed and adjusted and whose efficacy period can be controlled so that circadian disturbances that occur under dopamine therapy when treating dopaminergic diseases, in particular, when treating patients with Parkinson's disease, are prevented. The α-adrenolytic effect of lisuride and the ergoline derivatives of the formula I has another benefit for this application in that it also noticeably diminishes urinary urgency at nighttime and other bladder dysfunctions that are rather common in Parkinson patients (such as prostatic hyperplasia), which adds to the success of the therapy.

The technological problem is solved according to the invention in that a transdermal therapeutic system (TTS) is used comprising a pharmaceutical layer containing at least one matrix having an active ingredient, and/or an active ingredient reservoir; a diffusion barrier which is permeable to active ingredients and which is arranged on the skin side of the active ingredient reservoir; and an ergoline derivative according to formula I or physiologically compatible salt thereof with an acid,

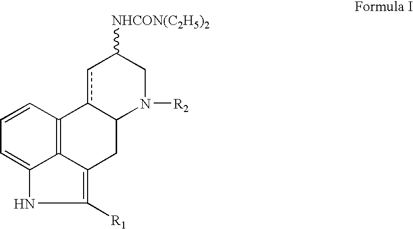

wherein ------ is a single or double bond wherein R1 is a H atom or a halogen atom, particularly a bromine atom, and wherein R2 is a C1-C4 alkyl, particularly methyl, as means of obtaining and maintaining the circadian rhythm under continuous dopamine therapy. Suitable salts of the active ingredients include sulfates, phosphates, maleates, citrates and succinates, especially hydrogen maleate.

The invention is based on the surprising finding that circadian disturbances under dopamine therapies can be prevented using an ergoline derivative of the formula I or a salt thereof that is highly effective and has a short half-life (0.5 to 4 hours, particularly 1 to 2 hours). A special benefit this invention offers is the establishment of a continuous active ingredient flux so that plasma concentrations can be set as defined and variations can be controlled. This mainly prevents the dopaminergic side effects such as fatigue, dizziness, etc. that are observed with single oral administrations or using a TTS containing an active ingredient with a long half-life. It was found that these side effects can be prevented when the level of active ingredient in the plasma is not subject to any major and rapid variation, an automatic occurrence with oral administration, but is set slowly and continuously. In addition, the problems encountered with oral administration such as greatly varying absorption rates and a not too well-defined time of maximum concentration in the plasma depending on the type and time of food intake are virtually eliminated by this invention. Most of all, it prevents overdosing (and thus REM suppression and other disruptions of the sleep pattern). Furthermore, administration can easily be canceled by just removing the TTS. The drop in agent concentration in the plasma when removing the TTS is further accelerated because of the short half-life of the suitable agents according to the invention. Unlike discontinuing an orally administered active agent or an active agent with a long half-life, decomposition in the plasma is fast and controlled, which also prevents a hangover. Finally it is easy to administer exact individual doses by selecting the flux F and/or the active surface area. It is preferred to select the flux F and the active surface area for reaching an effective dose in the range from 10 μg to 2 mg of active ingredient (such as lisuride), preferably 50 μg to 1 mg, throughout the day or over 24 hours in the patient's system on the second day of application.

It is further preferred to select the matrix and/or diffusion barrier so that the transdermal flux F through human skin measured as described in Example 1 is in the range from 0.1 to 5.0 μg/cm²/h, preferably 0.5 to 2.5 μg/cm²/h. A patch with these specifications is particularly suited for obtaining continuous lisuride concentrations in the plasma in the range from 0.05 to 5.0 ng/ml, preferably 0.1 to 0.5 ng/ml. The use of a TTS comprising a matrix and an ergoline derivative of the formula I or salt thereof as the active ingredient.

The list of ergoline derivatives that can be used includes the following: Bromolisuride (3-(2-bromo-9,10-didehydro-6-methyl-8α-ergolinyl)-1,1-diethyl urea), terguride (3-(6-methyl-8α-ergolinyl)-1,1-diethyl urea) and proterguride (3-(6-propyl-8α-ergolinyl)-1,1-diethyl urea). However it is preferred when the ergoline derivative is lisuride (3-(9,10-didehydro-6-methyl-8α-ergolinyl)-1,1-diethyl urea) or a physiologically compatible salt thereof with an acid. The production of lisuride and other suitable ergolines according to the invention is described, inter alia, in U.S. Pat. No. 3,953,454, EP 056 358 and U.S. Pat. No. 4,379,790. Suitable salts of the ergoline derivative include sulfates, phosphates, maleates, citrates and succinates, especially hydrogen maleate.

The TTS can be designed as follows. A covering layer can be arranged on the side of the matrix and/or active ingredient reservoir facing away from the skin. It may be formed by films of polyethylene or polyester. It is typically 10 to 100 microns in thickness. The covering layer may be pigmented and/or metal plated to ensure sufficient protection from light. Metal plating involves applying a very thin layer (typically less than 1 micron, mostly in the 10-100 nm range) of a metal such as aluminum to the covering layer. Pigments can be all pigments commonly used for coating including effect pigments as long as these are physiologically harmless. A detachable liner such as a siliconized or fluoropolymer-coated protective film can be provided on the application side.

The matrix and/or diffusion barrier may comprise as their main matrix component a substance selected from the group consisting of “polyacrylate, polyurethane, cellulose ether, silicone, polyvinyl compounds, silicate and mixtures of these substances as well as copolymers of these polymeric compounds,” preferably hydrophilic polyacrylate with basic substituents. A main matrix component makes up at least 50 percent by weight, e.g. at least 80-90 percent by weight of the matrix (matrix to be understood as the finished layer, i.e. main matrix component(s) with adjuvant(s) and active ingredient(s)). The desired flux is set by selecting the substance depending on the diffusion coefficient of the active ingredient and, if required, by selecting the layer thickness of the matrix in orthogonal direction to the skin surface. Matrix thickness is typically in the range from 10 to 500 microns.

A preferred polyacrylate adhesive as main matrix component is commercially available under the brand name GELVA® multipolymer solution 7881, provided by Monsanto Deutschland GmbH, Düsseldorf. We expressly refer to the product sold under this name and its datasheet in the version of Apr. 23, 1996. Eudragit® E100, provided by Röhm, Germany, is a copolymerisate from dimethyl aminomethyl methacrylate with neutral methacrylate esters and particularly well suited for use.

The polyacrylate adhesives listed above provide an advantageous non-trivial combination of properties, namely optimum flux, good adhesive power, good skin compatibility, and durability.

The diffusion barrier can alternatively comprise as its main barrier component a polymer selected from the group consisting of “cellulose ester, cellulose ether, silicone, polyolefin and mixtures as well as copolymers of these substances.” What has been said about the term of the main matrix component above analogously applies to the term of the main barrier component. The diffusion barrier can be a film with a thickness from 10 to 300 microns; the actual film thickness is selected (in conjunction with the diffusion coefficient of the active ingredient in the polymer) according to the desired flux.

The matrix and/or active ingredient reservoir and/or diffusion barrier may contain the common adjuvants used in TTSs. It is preferred to use a penetration-enhancing agent that is preferably selected from the group consisting of “C1-C8 aliphatic, cycloaliphatic and aromatic alcohols, saturated and unsaturated C8-18 fatty alcohols, saturated and unsaturated C8-18 fatty acids, hydrocarbons and hydrocarbon mixtures, fatty acid esters from C3-19 fatty acids and C1-6 alkyl monools, dicarboxylic acid dieesters from C4-8 dicarboxylic acids and C1-6 alkyl monools, and mixtures of these substances. Penetration-enhancing agents improve the flux of the active ingredient through the skin to which the TTS is attached. Examples of the substances listed above are: 1,2-propane diol, menthol, dexpanthenol, benzyl alcohol, lauryl alcohol, isocetyl alcohol, cetyl alcohol, mineral oil, lauric acid, isopalmitic acid, isostearic acid, oleic acid; methyl ester, ethyl ester, 2-hydroxyethyl ester, glycerol ester, propyl ester, isopropyl ester, butyl ester, sec. butyl ester or isobutyl ester of lauric acid, myristic acid, stearic acid, or palmitic acid. Use of dimethyl isosorbide, isopropyl myristate and lauryl alcohol is preferred, use of lauryl alcohol is most preferred. Other adjuvants are, for example, crystallization inhibitors. Suitable crystallization inhibitors are highly dispersed silicon dioxide or macromolecular substances such as polyvinyl pyrrolidone, polyvinyl alcohols, dextrines, dextranes, sterines, bile acids and, in particular, vinyl pyrrolidone vinylacetate copolymers such as Kollidon® VA 64. It goes without saying that the penetration-enhancing agent has to be able to diffuse to a sufficient extent through the matrix or diffusion barrier. If a matrix and lauryl alcohol as an adjuvant are used, it is preferred that the lauryl alcohol makes up 10 to 30 percent by weight, most preferred 15 to 20 percent by weight, of the matrix.

In addition to the ingredients listed above, sufficient quantities of sulfur-containing amino acids such as cysteine, methyl donors such as methionine, or antioxidants such as glutathione or sodium hydrogensulfite are added to the matrix as antioxidants because studies have surprisingly shown that this can prevent or dramatically reduce the formation of toxic oxidation products of lisuride such as lisuride-N-oxide. Antioxidants like glutathione can also have a synergistic effect on Parkinson's disease as oxidative stress plays an important part her; it has been known that even from early stages on there is a glutathione shortage in the dopaminergic substantia nigra. Methionine again is particularly desirable as a methyl donor because levodopa is mainly decomposed through oxygen methylation (COMT); homoserine levels increase due to the required levodopa quantities (daily dose up to the gram range), which is suspected to be a risk factor for cardial and cerebral events.

The adjuvants can basically make up from 0 to 50 percent by weight of the matrix. The active ingredient can make up 0.2 to 20 percent by weight, preferably 1 to 10 percent by weight, of the matrix. The sum total of main matrix component, adjuvants and active ingredients is always 100 percent by weight.

The active ingredient dose in a human body carrying a TTS is dependent on the diffusion-related properties of the TTS mentioned above and also on its active surface area on the skin. Active surface area means the area over which the matrix or diffusion barrier comes to rest on the skin. Variation in accordance with the desired dosage will preferably be in a range from 1 to 100 cm².

Within the scope of this invention, a physician can easily set up personalized dose variations for a flux adjusted to the given indication by selecting a suitable patch size. Thus the treatment can easily be adjusted to different body weights, age groups, etc. It is particularly feasible to equip a TTS comprising a (rather large) standard area with subdivision markers for partial doses so that a user can just remove the protective film from a partial area corresponding to the specified dose. The respective subsections can easily be printed on the covering layer.

The use of lisuride, its salts or derivatives with comparably favorable properties as active ingredients offers the following therapeutic benefits:

These substances can be applied at extremely low doses (for lisuride: from 0.075 mg orally at a high first pass effect) due to their extraordinarily strong affinity for dopamine and other monoamine receptors; thus a TTS with a relatively small application area can easily build an effective and well adjustable active ingredient level across the area over 24 hours or longer;

Unlike long-acting oral active ingredients such as cabergoline, transdermal dosing of lisuride not only is much improved (elimination of the considerable and highly variable first pass effect after oral administration of cabergoline or the like), the effects can also easily be discontinued whenever required (e.g. when side effects occur) by removing the patch. Then the short half-life of lisuride in the blood (ca. 2 hrs) comes in useful—a great contrast to oral dopamine agonists where side effects last for days once they are administered.

The combination of these effects has surprisingly resulted in combining the benefits of continuous and long-lasting dopaminergic stimulation with the other benefits of short-term acting dopaminergic pharmaceuticals in one application.

Combining these properties enables physicians to tailor the application to a patient's individual situation and needs as they can select the application scheme of two patches (simultaneous removal and reattachment, overlapping replacement or replacement at an interval) or, even better, to obtain almost any circadian rhythm of dopaminergic therapy by modifying the initial flux rate of the TTS formulation:

A Continuous stimulation when the initial flux rate of the patch matches the terminal half-life after patch removal (tmax−t/2, optionally a short interval, or when simultaneously applying a new TTS with a relatively high initial flux rate)

B A phase with enhanced stimulation (e. g. when adjusting the therapy or for bridging a patient's ‘off’ phase) by applying the second patch while the first is still attached to the skin or by using patches with a high initial flux rate (tmax<<t/2) or very low initial elimination rate (e. g. when the application area is small and the diffusion of the active ingredient increases with the decrease of the concentration gradient), and

C a phase of reduced dopaminergic stimulation such as reducing time-of-day-specific side effects by either complying with an interval between patch removal and attachment of the new patch, or, even simpler, by simultaneously using the new patch with a very low initial flux rate (tmax>>t/2) at the time of removal.

In all, we are surprisingly facing the chance of using just one active ingredient with suitable receptor affinity, efficacy and kinetics and opening all options of an easily applicable and well adjustable dopamine treatment for the patient. As the side effects that are almost inevitable when using state-of-the-art oral and transdermal therapies are prevented, stronger efficacy and a clearly improved therapeutic effect are obtained with simple means.

This means that levodopa therapy and its long-term complications can be prevented or delayed or that this or any other oral dopamine therapy has to be applied at low doses only and is thus more compatible.

In this context, the invention also includes a TTS set for obtaining and maintaining a continuous receptor stimulation with circadian rhythm, particularly for Parkinson's disease, said set containing multiple TTS elements that are set up for releasing different doses. The TTS elements can be separated for this purpose, each TTS element being configured for a continuously ascending sequence of F ranging from 0.1 to 5 μg/cm²/h. In addition, or separately, TTS elements can be equipped with a continuous sequence of differing active areas. In the latter case it is possible to use uniform F values. The TTS elements can be arranged on a big TTS design showing markings that indicate the areas to be used. An embodiment in which these elements are separated is conceivable as well, of course.

The invention can also be used for other indications. One application is the use of a TTS according to the invention to produce an agent for the treatment or prevention of the premenstrual syndrome or its symptoms, wherein F preferably is in the range from 0.1 to 0.5 μg/cm²/h, another one to produce an agent that inhibits lactation, wherein F preferably is in the range from 0.1 to 0.5 μg/cm²/h.

The invention will be explained in more detail below based on various examples.

### EXAMPLE 1

### Flux Measurement

A FRANZ flow-through diffusion cell is used for flux measurement. The measuring area is 2 cm². 4 cm² of ventral and dorsal skin of a male hairless mouse (MFl hr/hr Ola/Hsd, provided by Harlan Olac, UK) are used as our skin sample after carefully removing any subcutaneous fatty tissue. A 2 cm² TTS is applied to the skin sample. The acceptor medium is placed on the opposite side. It is diluted HHBSS (Hepes Hanks Balanced Salt Solution) containing 5.96 g/l of Hepes, 0.35 g/l of NaHCO₃ and 0.1 ml/l 10× of HBSS (provided by Gibco, Eggenstein, DE). Furthermore, 1000 I.U./ml of penicillin (benzylpenicillin potassium salt, provided by Fluka, Neu-Ulm, DE) are used.

The flux is measured as described below. First, the TTS to be measured is applied to the skin. The skin is mounted in the diffusion cell immediately thereafter. Samples of the acceptor medium are taken at 2-hour intervals between t=0 hrs and t=6 hrs and at 8-hour intervals between t=6 hrs and t=54 hrs. 1 ml of acceptor medium per hour is pumped through the diffusion cell using a peristaltic pump. The temperature of the acceptor medium is controlled using a circulating water bath which keeps the skin at a temperature of 31° C. with an accuracy of 1° C.

The active ingredient concentration in the acceptor medium is determined in accordance with the following specifications using a radioimmunoassay.

Calibration Curves:

These are constructed using two different methanol solutions of non-radioactive lisuride hydrogen maleate salt, each containing 1 mg/ml. These solutions are individually diluted with BSA buffer (0.041 M of Na₂HPO₂*2H₂O, 0.026 M of KH₂PO₄, 0.154 M of NaCl, 0.015 M of NaN₃, 0.1% (w/v) of BSA, pH 7, supplemented with 0.05% (w/v) of ascorbic acid) to obtain lisuride-free base concentrations in the range from 1000-3.9 pg/0.1 ml. In addition, a sample without active ingredient (0 pg) is used. The calibration samples are analyzed three times. The lisuride concentrations are calculated using the pharmacokinetic PC program RIO 2.5 (other common software may also be used).

Sample Preparation:

The acceptor medium is diluted with BSA buffer prior to the analysis to set the concentrations to an analyzable range of the calibration curve. 100 μl of diluted sample are directly subjected to radioimmunological analysis.

Antiserum:

The antiserum (rabbit) is obtained by immunizing with lisuride-1-succinyl-BSA, an immunogen. The antiserum in the assay is diluted 1:12500.

Tracer:

³H-lisuride hydrogen maleate with a specific activity of 4.3 GBq/mg is used.

Incubation:

0.1 ml of BSA buffer with active ingredient, 0.1 ml of tracer solution (ca. 5000 cpm/0.1 ml of BSA buffer) and 0.1 ml of diluted antiserum (1:12500) are added to 0.7 ml of BSA buffer and incubated for 18 hours at 4° C.

Separation:

antibody-bound lisuride is separated from free lisuride by adding 0.2 ml of charcoal suspension (1.25% (w/v) and 0.125% (w/v) of dextrane in BSA buffer) and incubation for 30 minutes at 0° C. The charcoal is sedimented by centrifuging for 15 minutes at 3000 g. The supernatant liquid (containing antibody-bound active ingredient) is decanted and subjected to radiometric analysis.

Radiometric Analysis:

4 ml of Atomlight (NEN) scintillation cocktail are added to the supernatant. The count is carried out using a WALLAC 1409 or 1410 β-scintillation counter without quench control.

Analysis:

The percutaneous skin flux is calculated as follows:

F=(C*R)/(A*T),

where F is the percutaneous flux [ng/cm²/h], C the active ingredient concentration in the acceptor medium [ng/ml], R the acceptor medium flow [1 ml/h], A the measured area [2 cm²] and T the sample-taking interval [h].

The maximum transdermal active ingredient flux is obtained directly from the data. Mean percutaneous flux values are determined during days 1 and 2 of the experiment based on the cumulative absorbed dose in time intervals t=0-22 and t=22-54.

Specifications for the Production of TTS

### EXAMPLE 2

### TTS A

15 mg of Kollidon VA 64 (crystallization inhibitor) are dissolved in 15 mg of isopropanol. Then 5 mg of lisuride are sprinkled in. 80 mg of polyacrylate adhesive (Gelva 7881) are placed in a beaker, and the above suspension is added while rerinsing with 30 mg of isopropanol. The crystal-free wet mix obtained is thoroughly intermixed and spread on a siliconized liner using a 500 micron blade. The product is dried at 60° C. for 20 minutes, and finally a covering layer is laminated onto it.

Flux measurements as described in Example 1 showed an F value of 0.43 on day 1, 0.44 on day 2, and a maximum F value of 0.85 (each in μg/cm²/h).

### EXAMPLE 3

### TTS B

12.5 mg of dimethyl isosorbide are suspended with 2 mg of lisuride in 15 mg of isopropanol. 80 mg of polyacrylate adhesive (Gelva 7881) are placed in a beaker, and the above suspension is added while rerinsing with 30 mg of isopropanol. The crystal-free wet mix obtained is thoroughly intermixed and spread on a siliconized liner using a 500 micron blade. The product is dried at 60° C. for 20 minutes, and finally a covering layer is laminated onto it.

Flux measurements as described in Example 1 showed an F value of 0.23 on day 1, 0.28 on day 2, and a maximum F value of 0.50 (each in μg/cm²/h)

### EXAMPLE 4

### TTS C

27.2 mg of polyvinyl pyrrolidone (crystallization inhibitor) and 16.3 mg of lauryl alcohol are dissolved at 60° C. Then 2 mg of lisuride and 0.5 mg of glutathione are dissolved in this solution at 60° C. 39.38 mg of Eudragit E100, 13.41 mg of Citroflex 4A and 1.71 mg of succinic acid are molten at 150-200° C. The lisuride solution is added after the batch has cooled down to 80° C. The product is spread at 80° C. on a siliconized liner using a 500 micron blade. Then the product is cooled down to 20° C.; optionally, a covering layer may be laminated onto it.

Flux measurements as described in Example 1 showed an F value of 0.90 on day 1, 1.6 on day 2, and a maximum F value of 2.4 (each in μg/cm²/h).

1) Use of a transdermal therapeutic system (TTS) comprising a pharmaceutical layer containing at least one matrix having an active ingredient, and/or an active ingredient reservoir; a diffusion barrier which is permeable to active ingredients and which is arranged on the skin side of the active ingredient reservoir; and an ergoline derivative according to formula I or physiologically compatible salt thereof with an acid, 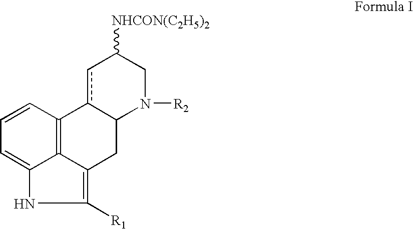
wherein ---- is a single or double bond wherein R1 is an H atom or a halogen atom, particularly a bromine atom, and wherein R2 is a C1-4 alkyl,
for producing an agent for obtaining and maintaining the circadian rhythm under a continuous dopamine therapy.

2) The use according to claim 1 wherein the matrix and/or diffusion barrier are selected so that the transdermal flux F through human skin is in the range from 0.1 to 5.0 μg/cm²/h.

3) The use according to claims 1 or 2 wherein the ergoline derivative is lisuride or a physiologically compatible salt thereof.

4) The use according to any one of claims 1 through 3 wherein a covering layer is provided on the side of the matrix and/or active ingredient reservoir that faces away from the skin.

5) The use according to any one of claims 1 through 4 wherein the matrix and/or diffusion barrier comprise as their main matrix component a substance selected from the group consisting of “polyacrylate, polyurethane, cellulose ether, silicone, polyvinyl compounds, silicate and mixtures of these substances as well as copolymers of these polymeric compounds,” preferably hydrophilic polyacrylate with basic substituents.

6) The use according to any one of claims 1 through 5 wherein the diffusion barrier comprises as its main barrier component a synthetic polymer selected from the group consisting of “cellulose ester, cellulose ether, silicone, polyolefin and mixtures as well as copolymers of these substances.”

7) The use according to any one of claims 1 through 6 wherein the matrix and/or the active ingredient reservoir and/or the diffusion barrier contain a penetration-enhancing agent that is preferably selected from the group consisting of “C1-C8 aliphatic, cycloaliphatic and aromatic alcohols, saturated and unsaturated C8-18 fatty alcohols, saturated and unsaturated C8-18 fatty acids, hydrocarbons and hydrocarbon mixtures, fatty acid esters from C3-19 fatty acids and C1-6-alkyl monools, dicarboxylic acid diesters from C4-8-dicarboxylic acids and C1-6 alkyl monools, and mixtures of these substances.

8. The use according to any one of claims 1 through 7 wherein the matrix and/or the active ingredient reservoir and/or the diffusion barrier contain a crystallization inhibitor that is selected from the group consisting of “highly dispersed silicon dioxide or macromolecular substances such as polyvinyl pyrrolidone, polyvinyl alcohols, dextrines, dextranes, sterines, bile acids and, in particular, vinyl pyrrolidone vinylacetate copolymers.”

9. The use according to any one of claims 1 through 8 wherein the matrix and/or active ingredient reservoir and/or diffusion barrier contain an antioxidant that is selected from the group consisting of “sulfur-containing amino acids such as cysteine, methyl donors such as methionine, or antioxidants such as glutathione or sodium hydrogensulfite.”

10) Use of a TTS according to any one of claims 1 through 9 to produce an agent for the treatment or prevention of premenstrual syndrome wherein the preferred F value is in the range from 0.1 to 0.5 μg/cm²/h.

11) Use of a TTS according to any one of claims 1 through 9 to produce an agent for lactation inhibition wherein the preferred F value is in the range from 0.1 to 0.5 μg/cm²/h.

12) A TTS set for the treatment of circadian disturbances under dopamine therapy wherein the set contains a multitude of TTS elements and wherein said elements are configured for releasing different doses.

13) A TTS set according to claim 12 wherein the TTS elements are separated and wherein each TTS element is configured for a continuously ascending sequence of F ranging from 0.1 to 5 μg/cm²/h.

14) The TTS set according to claims 12 or 13 wherein the TTS elements are equipped with different active surfaces in a continuous sequence.
